(19) United States
(12) Patent Application Publication
(10) Pub. No.: US 2025/0180457 A1
(43) Pub. Date: Jun. 5, 2025

(54) IMAGING WHITE BLOOD CELLS IN PRESENCE OF RED BLOOD CELLS

(71) Applicant: IDEXX Laboratories, Inc., Westbrook, ME (US)

(72) Inventors: Jeremy Hammond, Standish, ME (US); James W. Russell, North Yarmouth, ME (US); Jason J. Aguiar, Freeport, ME (US); Kayla Carpenter, Gorham, ME (US)

(21) Appl. No.: 18/961,590

(22) Filed: Nov. 27, 2024

Related U.S. Application Data
(60) Provisional application No. 63/604,604, filed on Nov. 30, 2023.

(51) Int. Cl. G01N 15/01; G01N 15/14; G01N 15/1433; G01N 33/49; G02B 21/36; G06V 20/69

(52) U.S. Cl. CPC G01N 15/01; G01N 15/1433; G01N 33/49; G02B 21/367; G06V 20/693; G01N 2015/016; G01N 2015/1486

(57) ABSTRACT

An apparatus includes at least one processor and at least one memory storing instructions. The instructions, when executed by the processor(s), cause the apparatus at least to perform: focusing an imaging device into a sample chamber; determining that the sample chamber contains a biological sample that includes red blood cells and white blood cells; and controlling the imaging device to capture a plurality of images. The images include images of a first number of fields of view containing a portion of the sample chamber, and/or images of a second number of fields of view containing a portion of the sample chamber. The second number is different from the first number, and the first number of fields of view and the second number of fields of view correspond to different sample chamber depth dimensions, and/or different ratios of volume of the diluent to volume of the biological sample.

### CROSS-REFERENCE TO RELATED APPLICATIONS

This application claims the benefit of, and priority to, U.S. Provisional Patent Application No. 63/604,604, filed on Nov. 30, 2023, the entire contents of which are hereby incorporated herein by reference.

### TECHNICAL FIELD

The present disclosure relates to imaging of white blood cells, and more particularly, to imaging of white blood cells in a sample chamber in the presence of red blood cells.

### BACKGROUND

Hematology analyzers can be utilized to count and identify blood cells. For example, hematology analyzers can detect and count different types of blood cells and can identify anomalies within blood samples.

### SUMMARY

In accordance with aspects of the present disclosure, an apparatus for analyzing a biological sample includes: at least one processor and at least one memory storing instructions. The instructions, when executed by the at least one processor, cause the apparatus at least to perform: focusing an imaging device into a sample chamber, where the sample chamber has a bottom wall that is at least translucent and through which the imaging device is configured to image an inside of the sample chamber; determining that the sample chamber contains at least a diluent and a biological sample that includes red blood cells and white blood cells; and controlling the imaging device to capture a plurality of images. The plurality of images includes at least one of: images of a first number of field of view containing at least a portion of the sample chamber, or images of a second number of fields of view containing at least a portion of the sample chamber, where the second number is different from the first number. The first number of fields of view and the second number of fields of view correspond to at least one of: different sample chamber depth dimensions, or different ratios of volume of the diluent to volume of the biological sample.

In accordance with aspects of the present disclosure, a method for analyzing a biological sample includes: focusing an imaging device into a sample chamber, where the sample chamber has a bottom wall that is at least translucent and through which the imaging device is configured to image an inside of the sample chamber; determining that the sample chamber contains at least a diluent and a biological sample that includes red blood cells and white blood cells; and controlling the imaging device to capture a plurality of images. The plurality of images includes at least one of: images of a first number of fields of view containing at least a portion of the sample chamber, or images of a second number of fields of view containing at least a portion of the sample chamber, where the second number is different from the first number. The first number of fields of view and the second number of fields of view correspond to at least one of: different sample chamber depth dimensions, or different ratios of volume of the diluent to volume of the biological sample.

In accordance with aspects of the present disclosure, a processor-readable medium stores instructions which, when executed by at least one processor of an apparatus, causes the apparatus at least to perform: focusing an imaging device into a sample chamber, where the sample chamber has a bottom wall that is at least translucent and through which the imaging device is configured to image an inside of the sample chamber; determining that the sample chamber contains at least a diluent and a biological sample that includes red blood cells and white blood cells; and controlling the imaging device to capture a plurality of images. The plurality of images include at least one of: images of a first number of fields of view containing at least a portion of the sample chamber, or images of a second number of fields of view containing at least a portion of the sample chamber, where the second number is different from the first number. The first number of fields of view and the second number of fields of view correspond to at least one of: different sample chamber depth dimensions, or different ratios of volume of the diluent to volume of the biological sample.

The details of one or more embodiments of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

A detailed description of embodiments of the disclosure will be made with reference to the accompanying drawings, wherein like numerals designate corresponding parts in the figures:

FIG. 1 is a diagram of example components of an apparatus or system, in accordance with aspects of the present disclosure;

FIG. 2 is an example of an image of red blood cells and a white blood cell, in accordance with aspects of the present disclosure;

FIG. 3 is an example of a fluorescence image a white blood cell, in accordance with aspects of the present disclosure;

FIG. 4 is a diagram of an example of an imaging device with illuminators, in accordance with aspects of the present disclosure;

FIG. 5 is a diagram of an example of portions of a sample chamber, in accordance with aspects of the present disclosure;

FIG. 6 is a diagram of an example of data relating to number of fields of view, in accordance with aspects of the present disclosure;

FIG. 7 is a flow diagram of an example of an operation for capturing images, in accordance with aspects of the present disclosure;

FIG. 8 is a diagram of an example of data relating to total time to count white blood cells, in accordance with aspects of the present disclosure;

FIG. 9 is a diagram of an example of a sample chamber, in accordance with aspects of the present disclosure; and

FIG. 10 is a diagram of an example of components of a point-of-care apparatus, in accordance with aspects of the present disclosure.

### DETAILED DESCRIPTION

The present disclosure relates to imaging of white blood cells (WBC) in a sample chamber in the presence of red blood cells (RBC).

As used herein, the term “exemplary” does not necessarily mean “preferred” and may simply refer to an example unless the context clearly indicates otherwise. Although the disclosure is not limited in this regard, the terms “plurality” and “a plurality” as used herein may include, for example, “multiple” or “two or more.” The terms “plurality” or “a plurality” may be used throughout the specification to describe two or more components, devices, elements, units, parameters, or the like.

As used herein, the term “approximately,” when applied to a value, means that the exact value may not be achieved due to factors such as, for example, manufacturing imperfections and/or wear over time, among other factors.

As used herein, the term “imaging device” refers to and means any device that is configured to sense at least the visible light spectrum and to provide an image. An imaging device may include components such as, without limitation, one or more lenses and a sensor.

As used herein, the term “field of view” refers to and means a region that is capturable by an imaging device. The term “working distance” refers to and means the object to lens distance where the image is at its sharpest focus. An image can be said to be focused on a scene at the working distance. The term “depth of field” refers to and means the distance between the nearest and furthest elements in a captured image that appear to be acceptably in focus. Depth of field and what is considered “acceptable” focus will be understood in the field of optical imaging. The term “resolving power” refers to the smallest distance between two features that an imaging device can clearly present as being separate features.

As used herein, the term “dilution ratio” refers to and means a ratio of volume of diluent to volume of biological sample. Accordingly, a ratio of volume of diluent to volume of biological sample of 75:1 may be described as a dilution ratio of 75:1. A diluent may be and include any substance or combination of substances that can be combined with a biological sample, including, without limitation, reagents, stains, buffers, and/or working fluids, among other possible substances.

Flow cytometers and impedance analyzers commonly separate whole blood analyses into two separate subsamples. The first subsample will incorporate reagents that cause RBC to change shape from a biconcave disk to a sphere usually by a function of osmolality and surfactant. RBC and platelets (PLT) are commonly assessed during this sequence. The second subsample will incorporate reagents that lyse RBC (cause them to pop open and release the hemoglobin trapped within) and may stain white cells (e.g., nucleic acid stains). WBC and platelets are commonly assessed during this sequence.

These automated hematology analyzers separate the sample because of the vast difference in normal range for each of the cell types, as shown in Table 1 below. The reference intervals define the natural range in which a sample for the species is expected to fall. RBC are found on the order of millions per microliter of sample, PLT are found on the order of hundreds of thousands per microliter of sample, and WBC are found on the order of thousands to tens of thousands per microliter, so the relative prevalence of each is vastly different.

TABLE 1
Reference intervals for various species

| Parameter | Human | Canine | Feline | Equine |
|---|---|---|---|---|
| RBC | (4.64-6.03 M/ul) | (5.65-8.87 M/ul) | (6.54-12.2 M/ul) | (6.4-10.4 M/ul) |
| PLT | (176-408 K/ul) | (148-484 K/ul) | (151-600 K/ul) | (100-250 K/ul) |
| WBC | (4.34-10.15 K/ul) | (5.05-16.76 K/ul) | (2.87-17.02 K/ul) | (4.9-11.1 K/ul) |

Analysis of whole blood can provide technical difficulties since there are generally 1,000 red blood cells (RBC) for every white blood cell (WBC) in the sample. If a significant number of WBC are needed in order to determine the WBC subtypes (called the differential), then there will be a large number of total cells to evaluate. In flow cytometry and impedance analyses, this problem is addressed by separating a run into two segments—the first to identify RBC, and the second to identify WBC after removing RBC from the sample. Splitting the sample into a RBC and a WBC analysis is effective but requires two sets of reagents-one for each analysis.

Manual microscope evaluation is another approach for whole blood analysis. Microscope evaluation may provide different information (and possibly more information) about the cells than flow cytometry, such as morphologies of particular cells. When evaluating whole blood samples, it is important to be able to differentiate RBC, PLT, and WBC, and, if possible, to evaluate the five types of WBC that make up the differential, including neutrophils, lymphocytes, monocytes, eosinophils, and basophils. There are normal ranges for each of this five-part differential, and eosinophils and basophils can normally be nearly zero and are important to evaluate when they are elevated. For basophils, elevated levels can occur at very low percentage values of the total WBC value, requiring many WBC to be sampled in order to determine proportional values for each of the differential parameters with some statistical confidence. However, manual counts tend to only evaluate 100 or 200 cells, with 400 cells being a large number to manually count. This range reflects what is practical for human clinicians to manually count. The statistical confidence with only a few hundred cells is poor when evaluating cells that are less than 10% of the sample.
Aspects of the present disclosure relate to automated evaluation of a blood sample based on capturing images of the blood sample by an imaging device and analyzing the captured images. In aspects, the present disclosure evaluates RBC and WBC in the same analysis in order to reduce a clinician's workflow, reagent management, and run costs. Aspects of the present disclosure relate to maximizing the number of WBC in a field of view while not having so many RBC in the field of view that the image is crowded and cannot be properly analyzed. Aspects of the present disclosure relate to completing the evaluation in a reasonable amount of time and with a reasonable degree of statistical confidence. As described below, various aspects of the present disclosure are implemented by combining a blood sample with a diluent and evaluating the combination in a sample chamber. The evaluation may capture a different number of images depending on the sample chamber depth dimension and/or the ratio of volume of diluent to volume of blood sample.

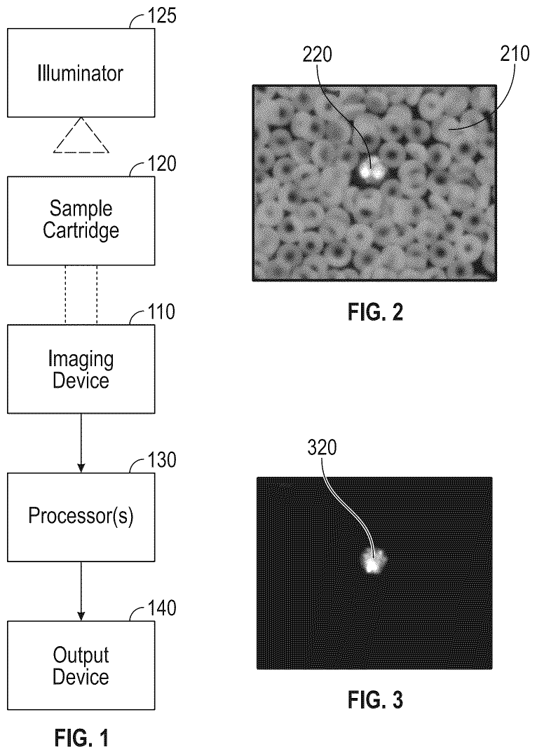

Referring to FIG. 1, a block diagram of various components is shown. The components include an imaging device 110, a sample cartridge 120, an illuminator 125, one or more processor(s) 130, and an output device 140. In embodiments, the components 110-140 may be co-located in a single apparatus, such as in a point-of-care apparatus of a veterinary clinic. In embodiments, one or more of the components may be located in different systems or devices. For example, one or more of the processor(s) 130 may be a processor in a cloud system. In embodiments, the components may include a communication device (not shown) for communicating information between different systems or devices.

The imaging device 110 is configured to capture a field of view containing at least a portion of the sample cartridge 120. In particular, the sample cartridge 120 includes a sample chamber, and the imaging device 110 captures a field of view containing at least a portion of the sample chamber. The sample chamber is illuminated by the illuminator 125, which may include a brightfield illuminator and/or a light source which induces Stokes shift and causes stained WBCs to fluoresce. An example of a light source which induces Stokes shift and causes stained WBCs to fluoresce is a blue light LED (light emitting diode) or an ultraviolet light LED. Such light sources may be implemented using a brightfield illuminator together with one or more filters configured to pass only blue light. Known staining methods may be used in order to cause the WBCs to fluoresce. In embodiments, the illuminator 125 may be positioned relative to the sample cartridge 120 in different ways and may include separate components that have different positions relative to the sample cartridge.

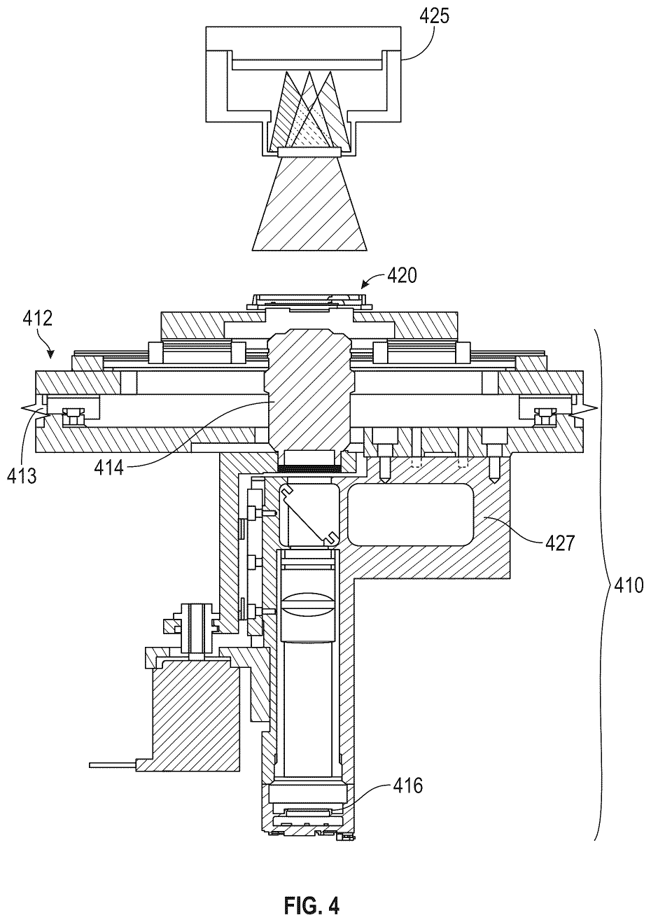

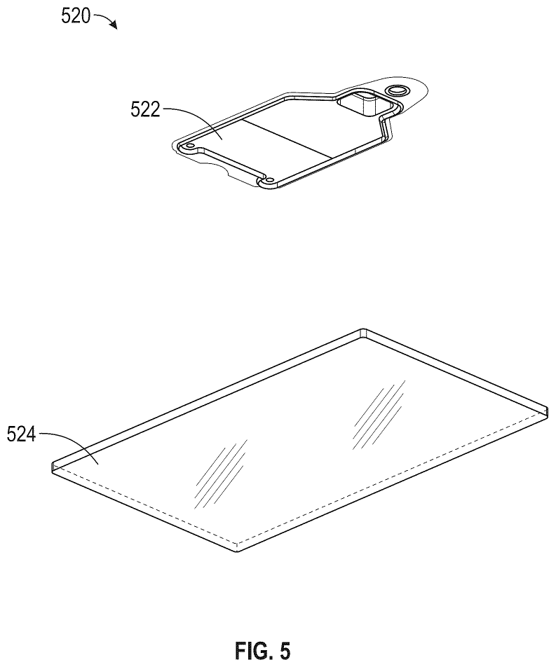

In embodiments, the sample cartridge 120 is movable to enable the imaging device 110 to capture different fields of view containing at least a portion of the sample chamber. In embodiments, rather than the sample cartridge 120 moving, the imaging device 110 is movable to capture different fields of view containing at least a portion of the sample chamber. An example of the imaging device 110 will be described in more detail in connection with FIG. 4, and an example of the sample cartridge 120 will be described in more detail in connection with FIG. 5.

Capturing multiple fields of view will be described in more detail later herein. For now, it is sufficient to note that each field of view captures at least a portion of the sample chamber of the sample cartridge 120. In embodiments, an entire cross-sectional area of a sample chamber of the sample cartridge 120 corresponds to more than one-hundred fields of view, such as in the range of two-hundred to four-hundred fields of view, among other possibilities. In embodiments, the entire cross-sectional area of the sample chamber in the sample cartridge 120 corresponds to less than one-hundred fields of view. In embodiments, the imaging device 110 and/or the sample cartridge 120 move at a rate such that between fifty to one-hundred different fields of view are captured each minute. In embodiments, other rates of capturing fields of view are within the scope of the present disclosure.

With continuing reference to FIG. 1, the processor(s) 130 are configured to analyze the images captured by the imaging device 110. As an example, in embodiments where the illuminator 125 is a brightfield illuminator, any RBCs in the images would appear in color and any WBCs in the images would appear in a different color. As another example, in embodiments where the illuminator 125 is a light source that induces Stokes shift and causes any stained WBCs to fluoresce without causing any RBCs to fluoresce, the images would only show the fluorescing WBCs. In embodiments, the illuminator 125 contains a brightfield illuminator as well as a light source that causes fluorescence. In embodiments, brightfield images and fluorescence images may be combined to provide composite images.

FIG. 2 shows an example of a composite image generated based on multiple brightfield and fluorescent images and is shown in grayscale rather than in color. In the image of FIG. 2, red blood cells 210 appear in one color (e.g., red), while a white blood cell 220 appears in another color (e.g., bluish). In embodiments such as FIG. 2, the processor(s) 130 may identify WBCs in the images by their color and/or by their shape. Various ways of doing so include without limitation, applying one or more trained machine learning models (e.g., convolutional neural network) that are trained to perform object detection to detect WBCs and/or detect various types of WBCs, i.e., neutrophils, lymphocytes, monocytes, eosinophils, and/or basophils.

FIG. 3 shows an example of a single fluorescent image (shown in grayscale) in which RBC do not fluoresce, so the image shows only the fluorescing WBC 320 but no RBCs. In embodiments such as FIG. 3, the processor(s) 130 may identify WBCs in the images by their mere presence and/or by their shape. Various ways of doing so include, without limitation, applying one or more trained machine learning models (e.g., convolutional neural network) that are trained to perform object detection to detect WBCs and/or detect various types of WBCs, i.e., neutrophils, lymphocytes, monocytes, eosinophils, and/or basophils.

The examples of FIG. 2 and FIG. 3 are merely illustrative. In embodiments, other constituents (e.g., platelets, etc.) may be imaged by brightfield imaging and/or fluorescence imaging, and machine learning models and/or image analytics may be applied to detect one or more constituents in such images. In embodiments, brightfield images and fluorescence images may be combined to provide composite images. In embodiments, other types of images are contemplated to be within the scope of the present disclosure.

With continuing reference to FIG. 1, the processor(s) 130 count number of WBCs in the images and, optionally, may count number of RBCs or platelets in the images, among other possible constituents. In embodiments, the processor(s) 130 may also apply machine learning models and/or image analytics to detect RBCs, platelets, and/or other constituents. In embodiments, the processor(s) 130 may also apply machine learning models and/or image analytics to detect various WBC morphologies, such as left shift (immature neutrophils), toxic change, reactive lymphocytes, among other morphologies. Known methods can be used to implement such machine learning models and/or image analytics.

The processor(s) 130 causes the output device 140 to provide information regarding WBCs to a person or user, such as number of WBCs counted and/or detected WBC morphologies, among other possible WBC information. The output device 140 may be any output device capable of communicating information to a person or user. In embodiments, the output device 140 is a display panel of a point-of-care device and is in the same device as the other components 110-130. In embodiments, the output device 140 may be an office computer or smartphone of a clinician, and a network device (not shown) may communicate the WBC information to the office computer or smartphone for display. For example, the processor(s) 130 may cause a text message or an email, that contains the WBC information, to be sent, and the output device 140 may receive and display the text message or email to a user. Other types of output devices 140 are contemplated to be within the scope of the present disclosure, such as audio output devices, among other possibilities.

FIG. 1 is merely an example of some components of an apparatus. It should be understood that an apparatus will include other components not shown in FIG. 1, such as a power supply, memory, electronic storage, network device, and/or other components. Such components, and other variations of an apparatus, are contemplated to be within the scope of the present disclosure.

Referring now to FIG. 4, there is shown an example of an imaging device 410, a sample cartridge 420, a brightfield illuminator 425, and one or more light sources 427 that induce Stokes shift. In the illustrated embodiment of FIG. 4, portions of the imaging device 410 and of the light source(s) 427 are integrated into a common housing. The brightfield illuminator 425 illuminates the sample cartridge 420 from above, and the light source(s) 427 illuminate that sample cartridge 420 from below. The brightfield illuminator 425 and the light source(s) 427 are controlled by a processor (e.g., 130, FIG. 1) to turn on or off at desired times to illuminate the sample cartridge 420. In embodiments, the light source(s) 427 may be separate from the imaging device 410. In embodiments, the brightfield illuminator 425 and the light source(s) 427 may be positioned and oriented differently than as shown in FIG. 4.

The imaging device 410 includes a positioning mechanism for positioning the sample cartridge 420 above a camera lens assembly 414 of the imaging device 410. The camera lens assembly 414 includes at least one lens and has a configured field of view, depth of field, resolving power, and magnification, among other characteristics. In embodiments, the camera lens assembly 414 provides a fixed optical magnification, such as 10×, 20×, or 40× optical magnification or another optical magnification, which enables the imaging device 410 to function as a microscope. In embodiments, the camera lens assembly 414 provides an adjustable magnification.

The positioning mechanism includes a platform 412 and includes motors 413 which move the platform 412. In the illustrated embodiment, the camera lens assembly 414 is stationary, and the positioning mechanism is capable of moving the sample cartridge 420 in two or three orthogonal directions (e.g., X and Y directions, optionally Z direction) to enable the camera lens assembly 414 to capture different fields of view containing at least a portion of the sample cartridge 420. The X- and Y-directions support moving to different fields of view, and the Z-direction supports changes to the depth level at end of the working distance.

Light from the field of view captured by the camera lens assembly 414 is directed to a sensor 416 through various optical components, such as a dichroic mirror and a lens tube, among other possible optical components. The sensor 416 may be a charge coupled device that captures light to provide images. The captured images are then conveyed to one or more processor(s) (e.g., 130, FIG. 1) for processing, as described in connection with FIG. 1.

As shown in FIG. 4, the imaging device 410 is below the sample cartridge 420. When the sample cartridge 420 contains a biological sample in a diluent, gravity causes constituents in the biological sample to settle in the sample cartridge 420 over time. Imaging the sample cartridge 420 from below allows the settling dynamics of various constituents to be used in evaluating the biological sample. For example, in certain diluents, WBCs settle at a rate greater than 1.0 micrometers per second, RBCs settle at a rate of about 1 micrometer per second, and platelets settle at a rate of about 0.3 micrometers per second. In the embodiment of FIG. 4, the sample cartridge 420 has a translucent or transparent bottom through which the image device 410 may capture images of fields of view containing at least a portion of the sample cartridge 420. The relative settling rates of the cells allow WBCs, which settle quicker, to separate to some degree from some of the RBCs and platelets in the sample cartridge over time and allow the imaging device 410 to capture images of WBCs more clearly as they settle.

FIG. 5 shows a bottom perspective view of an example of portions of a sample cartridge. The sample cartridge includes a sample chamber top portion 522 and a sample chamber bottom portion 524. The sample chamber top and bottom portions 522, 524 combine together to form a sample chamber between them, with an inlet port 526. The bottom sample chamber portion 524 has a translucent or transparent bottom through which an imaging device can image the sample chamber. In embodiments, the bottom sample chamber portion 524 is made of glass. In embodiments, the bottom sample chamber portion 524 can be made of polymers as long as they are optically clear, are flat, and do not auto fluoresce. The sample cartridge may have any suitable shape and dimensions for interoperability with an imaging device and/or with a point-of-care apparatus. The sample chamber formed by the top and bottom portions 522, 524 may have any suitable shape and dimensions for holding a biological sample and other materials, such as reagents and/or diluents, among other possible materials.

In embodiments, the sample chamber is configured to have a sufficient depth dimension to allow constituents in the sample to separate to some degree as they settle in the sample chamber. In embodiments, the sample chamber has a single depth dimension throughout the sample chamber. In embodiments, the sample chamber has two or more regions that have different depth dimensions. For example, as illustrated, the sample chamber may include a flat bottom portion 524 and a molded top portion 522 that creates the regions with different depth dimensions. In embodiments, the sample chamber can include, for example, a region with a depth dimension of one-hundred (100) micrometers and a region with a depth dimension of three-hundred (300) micrometers. In embodiments, the sample chamber can include, for example, a region with a depth dimension of one-hundred (100) micrometers, a region with a depth dimension of two-hundred (200) micrometers, and/or a region with a depth dimension of four-hundred (400) micrometers. Sample chambers having regions with other depth dimensions and/or with other combinations of depth dimensions are contemplated to be within the scope of the present disclosure.

As mentioned above, in embodiments, an entire cross-sectional area of the sample chamber corresponds to more than one-hundred fields of view, such as in the range of two-hundred to four-hundred fields of view, among other possibilities. In embodiments, the entire cross-sectional area of the sample chamber in the sample cartridge corresponds to less than one-hundred fields of view.

Accordingly, various aspects of components of the present disclosure have been described with respect to FIGS. 1-5. As described above, aspects of the present disclosure evaluate RBCs and WBCs in the same analysis in order to reduce a clinician's workflow, reagent management, and run costs. In embodiments, the biological sample is a whole blood sample that is collected in EDTA (ethylenediaminetetraacetic acid) anticoagulant and is mixed with reagents which dilute the sample, maintain the morphologic properties of the sample, and stain cells containing nucleic acids. Other biological samples and collection techniques, reagents, and/or stains are contemplated to be within the scope of the present disclosure.

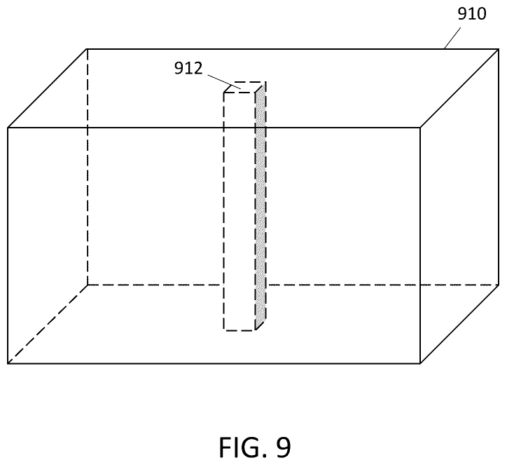

FIG. 9 shows a diagram of an example of a sample chamber 910. The shape and relative dimensions are merely illustrative, and other shapes and relative dimensions are contemplated. As mentioned above, in embodiments, an entire cross-sectional area of the sample chamber 910 corresponds to more than one-hundred fields of view of an imaging device (e.g., 410, FIG. 4), such as in the range of two-hundred to four-hundred fields of view, among other possibilities.

In the sample chamber 910 of FIG. 9, the column 912 corresponds to a single field of view, e.g., of an image device (e.g., 410, FIG. 4). A field of view containing a portion of a sample chamber may be captured using any working distance and depth of field. As mentioned above in connection with FIG. 4, each image captures a field of view. Working distance refers to the object to lens distance where the image is at its sharpest focus, and depth of field reflects the distance between the nearest and furthest elements in a captured image that appear to be acceptably in focus. For example, if a sample chamber depth dimension is one-hundred micrometers and the working distance is within the sample chamber, then a depth of field of ten micrometers would result in some depths within the sample chamber not being in acceptable focus in the field of view. In such cases, the lens of the imaging device and/or the sample chamber would need to be moved in the depth direction (e.g., Z-direction) to bring other portions of the depth within the sample chamber into acceptable focus.

An imaging device and/or a sample cartridge may move at a rate such that between fifty to one-hundred different fields of view are captured each minute. Other rates of capturing fields of view are contemplated.

As mentioned above, there is on average 1,000 RBCs for each WBC in a sample. The difference is even greater for particular types of WBCs, such as eosinophils and basophils, which can occur at very low percentage values of the total WBC value, requiring many WBC to be sampled in order to determine proportional values for each of the differential parameters with some statistical confidence.

When a biological sample and a diluent are properly mixed, the constituents (e.g., cells) will initially be well-distributed throughout the mixture; i.e., the density of the constituents will be similar throughout the mixture. Over time, various constituents will settle and come to rest at the bottom of the sample chamber. For a properly mixed (uniformly dense) mixture filled into two regions, a region having a larger depth dimension will contain a greater number of constituents per column of mixture than another region having a smaller depth dimension. Over time, the bottom of the region having larger depth dimension will be more crowded with constituents than the bottom of the region having a smaller depth dimension, due to a larger total number of constituents per column of mixture settling to the bottom.

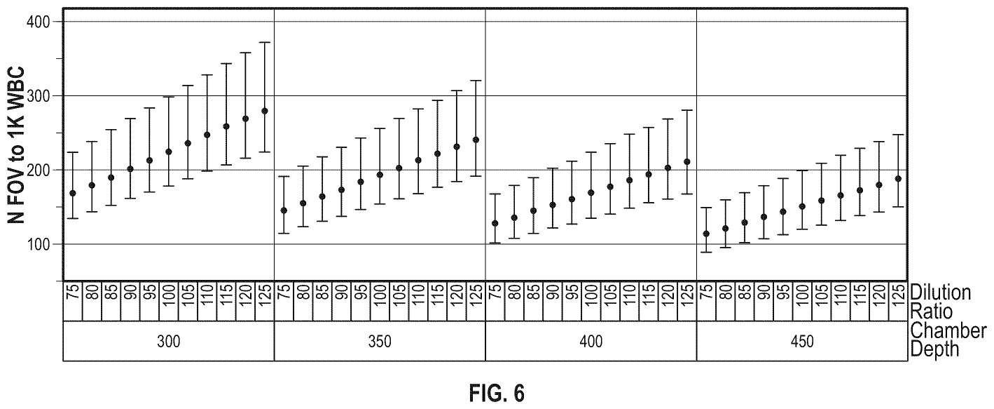

In accordance with aspects of the present disclosure, the number of fields of view that need to be captured by an imaging device to reach a statistically confident count of white blood cells (e.g., at least 1,000 WBCs) corresponds to the depth dimension of the sample chamber (or of the sample chamber region) that contains the white blood cells. FIG. 6 shows an example of data relating to counting white blood cells using the arrangement shown in FIG. 4. As shown in FIG. 6, for a particular ratio of volume of diluent to volume of biological sample (referred to as “dilution ratio”), the requisite number of WBCs may be counted using a fewer number of fields of view when the depth dimension is greater. For example, for dilution ratio of 75:1, the number of fields of view needed to count 1,000 WBCs in a 300 micrometer depth region is in the range of about 135-225 fields of view. In comparison, for the same dilution ratio of 75:1, the number of fields of view needed to count 1,000 WBCs in a 450 micrometer depth region is in the range of about 90-150 fields of view.

Also, as shown in FIG. 6, the number of fields of view that need to be captured by an imaging device to reach a statistically confident count of white blood cells (e.g., at least 1,000 WBCs) corresponds to dilution ratio of the mixture that contains the white blood cells. For a particular depth dimension, the requisite number of WBCs may be counted using a fewer number of fields when the dilution ratio is smaller. For example, for a 300 micrometer depth region, the number of fields of view needed to count 1,000 WBCs given a dilution ratio of 125:1 is in the range of about 220-375 fields of view. In comparison, for the same 300 micrometer depth region, the number of fields of view needed to count 1,000 WBCs given a dilution ratio of 75 is in the range of about 135-225 fields of view.

As demonstrated by the example data of FIG. 6, an imaging device does not need to capture the same number of fields of view for chambers or regions with different depth dimensions and/or for different dilution ratios. While the data of FIG. 6 is merely an example, the trends shown by the example of FIG. 6 will persist for different biological samples that settle. While the data of FIG. 6 shows a general trend of fewer fields of view for greater depth dimensions and lower dilution ratio, the data also reflects a range of fields of view for each combination of depth dimension and dilution ratio. The ranges of fields of view for different combinations of depth dimensions and dilution ratios may overlap, such that in various scenarios, number of fields of view may be greater for greater depth dimensions and/or for lower dilution ratios and may be smaller for lower depth dimensions and/or for greater dilution ratios. Such embodiments are within the scope of the present disclosure.

Generally, the number of fields of view depends on the concentration of the constituents to be identified and the number of constituents to be counted. The number of fields of view can be determined based on a depth dimension and a dilution ratio that are optimized for the number of constituents to be identified and counted and for their nominal concentrations. However, if the operation looks for different constituents in a shallower region than in a deeper region, with a higher nominal concentration in the shallower region, it is possible that more or fewer fields of view may be captured in the deeper region than in the shallower region. Such embodiments are within the scope of the present disclosure.

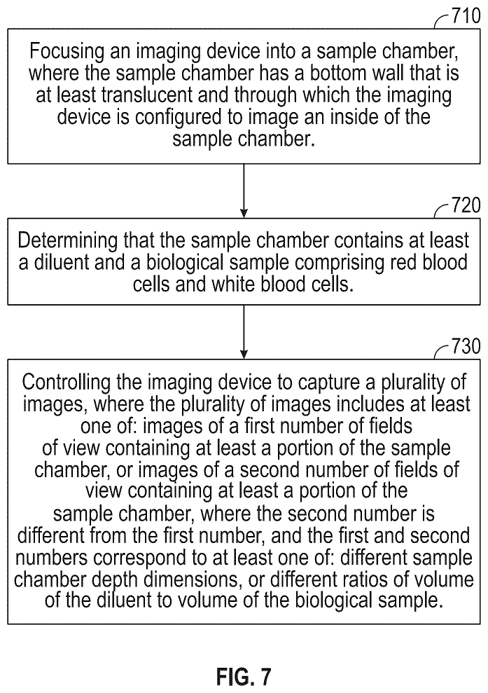

In accordance with aspects of the present disclosure, FIG. 7 is a flow diagram of an operation for evaluating a biological sample containing WBCs and RBCs. At block 710, the operation involves focusing an imaging device into a sample chamber, where the sample chamber has a bottom wall that is at least translucent and through which the imaging device is configured to image an inside of the sample chamber. In embodiments, the imaging device may be the imaging device 410 of FIG. 4 or may be another imaging device. In embodiments, the sample chamber may be the sample chamber of FIG. 5 or may be another sample chamber.

At block 720, the operation involves determining that the sample chamber contains at least a diluent and a biological sample comprising red blood cells and white blood cells. The contents of the sample chamber may be determined in various ways. In embodiments, the contents of the sample chamber may be determined by scanning a code on a sample cartridge, such as scanning a barcode or an alphanumeric code, among other possible codes. The scanner for scanning the code may be fixed within a point-of-care apparatus or may be a separate device, such as a camera of a smartphone, which can communicate a result of the scan to a point-of-care apparatus. In embodiments, a user of a point-of-care apparatus may manually indicate the contents of the sample chamber, e.g., by selecting an option on a user interface. Such and other embodiments are contemplated to be within the scope of the present disclosure.

At block 730, the operation involves controlling the imaging device to capture a plurality of images, where the plurality of images includes at least one of: images of a first number of fields of view containing at least a portion of the sample chamber, or images of a second number of fields of view containing at least a portion of the sample chamber, where the second number is different from the first number, and where the first and second numbers correspond to different sample chamber depth dimensions and/or different ratios of volume of the diluent to volume of the biological sample. That is, the imaging device captures different numbers of fields of view depending on the sample chamber depth dimension(s) and/or the dilution ratio, as exemplified by the data of FIG. 6.

As described in connection with FIG. 5 above, a sample chamber may have a single depth dimension throughout the sample chamber. In such embodiments, the operation of block 730 captures a number of fields of view corresponding to the depth dimension and the dilution ratio.

As described above, in embodiments, a sample chamber may include two regions that have different depth dimensions. In such embodiments, the operation of block 730 captures images of a first number of fields of view for a first region of the sample chamber having a first depth dimension and captures images of a second number of fields of view for a second region of the sample chamber having a second depth dimension. If the dilution ratio is the same in the two regions, the first and second numbers of fields of view correspond to the different sample chamber depth dimensions and to the dilution ratio. For example, in cases when the second depth dimension is greater than the first depth dimension, the second number of fields of view may be less than the first number of fields of view. If the dilution ratio is different in the two regions, the first and second numbers of fields of view correspond to the different sample chamber depth dimensions and to the different dilution ratios.

As described above, in embodiments, a sample chamber may include more than two regions that have different depth dimensions, such as three regions that have different depth dimensions. In such embodiments, the operation of block 730 captures a first number of fields of view for a first region of the sample chamber having a first depth dimension and captures a second number of fields of view for a second region of the sample chamber having a second depth dimension. Additionally, in the example of three regions, the operation of FIG. 7 captures a third number of fields of view for a third region of the sample chamber having a third depth dimension. If the dilution ratio is the same in the three regions, the first, second, and third numbers of images correspond to the different sample chamber depth dimensions and to the dilution ratio. For example, in cases when the third depth dimension is greater than the second depth dimension, which is greater than the first depth dimension, the third number of fields of view may be less than the second number of fields of view, which may be less than the first number of fields of view. If the dilution ratio is different in the three regions, the first, second, and third number of fields of view correspond to the different sample chamber depth dimensions and to the different dilution ratios. In embodiments, the first number of fields of view may be captured before capturing the second number of fields of view, which may be captured before capturing the third number fields of view. This approach allows constituents in the regions having greater depth dimensions to settle more or to fully settle before they are imaged.

For four or more regions in a sample chamber, the same principles described above apply to such sample chambers.

In aspects of the present disclosure, the captured images may include brightfield images and/or fluorescence images. In embodiments, brightfield images and fluorescence images are captured in a region. For example, multiple fluorescence images are captured in a higher depth dimension region (e.g., 300 micrometers or 400 micrometers) to optically erase the RBC and provide images showing WBC. Then, brightfield images can be captured only in the fields of view where a fluorescent image indicated a WBC is present. In embodiments, one or more regions may be captured using brightfield images, and one or more regions may be captured using fluorescence images. For example, brightfield images may be captured in regions where constituents would be less crowded, such as in smaller depth dimensions (e.g., 100 to 200 micrometers), and fluorescence images may be captured in regions where constituents may be more crowded, such as in larger depth dimensions (e.g., 200 micrometers or greater). An example is described farther below herein.

In accordance with aspects of the present disclosure, the fields of view captured by the operation of FIG. 7 may by any fields of view containing at least a portion of a sample chamber and may be captured using any working distance and depth of field. As mentioned above in connection with FIG. 4, each image captures a field of view. Working distance refers to the object to lens distance where the image is at its sharpest focus, and depth of field reflects the distance between the nearest and furthest elements in a captured image that appear to be acceptably in focus. For example, if a sample chamber depth dimension is one-hundred micrometers and the working distance is within the sample chamber, then a depth of field of one-hundred micrometers or larger would result in all depths within the sample chamber being in acceptable focus in the field of view. Depth of field and what is considered “acceptable” focus will be understood in the field of optical imaging. In contrast, if the depth of field is less than one-hundred micro-meters, then some depths within the sample chamber would not be in acceptable focus in the field of view. In the latter case, the lens of the imaging device would need to be moved in the depth direction (e.g., Z-direction) to bring other portions of the depth within the sample chamber into acceptable focus. In the former case, because the entire depth within the sample chamber is in acceptable focus, the imaging device and/or the sample cartridge may be moved in the non-depth directions (e.g., X- and Y-directions) to capture different constituents.

In embodiments, the operation of block 730 in FIG. 7 captures at least the bottom of the sample chamber in acceptable focus. In embodiments, the first number of images and the second number of images captured in block 730 include images focused on a plurality of depths in the sample chamber. For example, for a depth of field of twenty micrometers and a sample chamber depth dimension of one-hundred micrometers, images may be captured by focusing on five depths in the sample chamber, such as focusing on depths of 10, 30, 50, 70, and 90 micrometers. Such images focused on the five depths would capture an entire column of a 100-micrometer sample chamber.

As mentioned above, an imaging device and/or a sample cartridge may move at a rate such that between fifty to one-hundred different fields of view are captured each minute. In embodiments, other rates of capturing fields of view are contemplated. Based on the rate of capturing fields of view and the optical parameters (e.g., working distance, depth of field), it is possible to capture a sequence of field of views to count a requisite number of white blood cells (e.g., 1,000 WBCs) within a reasonable time period. In the case of a point-of-care apparatus in a veterinary facility, the amount of time deemed reasonable for acquiring a sample, preparing it in a sample cartridge, and analyzing the sample to provide a result, may vary, but a typical veterinarian visit may last no longer than thirty minutes. Therefore, as part of such a visit, a reasonable time period for a point-of-care apparatus to capture images of a sample chamber and analyze them may be less than ten minutes, for example. Counting WBCs involves using object detection and/or image analytics, as described above. When a requisite number of WBCs has been counted (e.g., 1,000 WBCs), the imaging operation may be stopped.

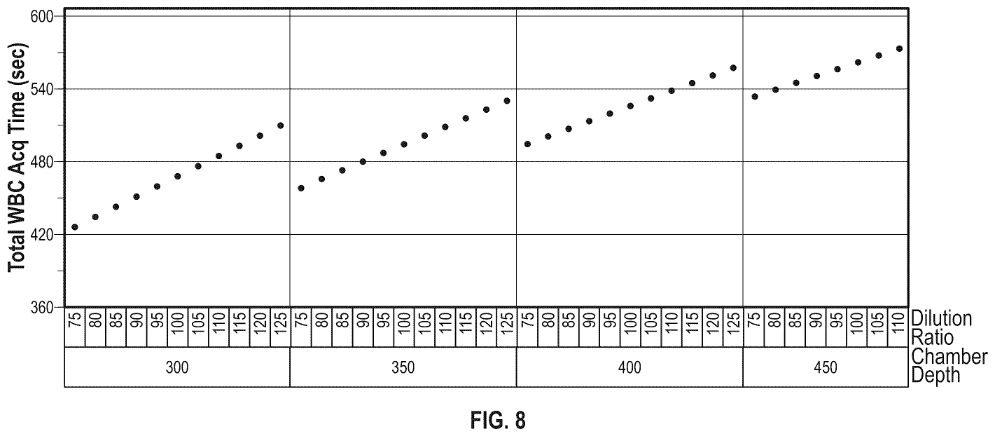

As an example of amount of time needed to conduct the counting, FIG. 8 shows an example of data relating to total time needed for cells to settle to the bottom of a chamber and to count 1,000 WBCs using the arrangement shown in FIG. 4.

For a chamber depth dimension of 300-micrometers, a constituent settling at 1-micrometer per second will need a settling time of 5 minutes to fully settle. For a chamber depth dimension of 450-micrometers, a constituent settling at 1-micrometer per second will need a settling time of 7 minutes 30 seconds to fully settle. Full settling time can be computed for other channel depth dimensions and other settling rates. Together with full selling time, additional time is required to capture the various fields of view to count 1,000 WBCs, and such additional time varies by dilution ratio. Generally, less time and fewer fields of view are needed to count 1,000 WBCs when the dilution ratio is lower, due to higher density of constituents, and more time and more fields of view are needed to count 1,000 WBCs when the dilution ratio is higher, due to lower density of constituents.

The data of FIG. 8 shows an example of allowing a constituent of interest to fully settle and counting 1,000 WBCs. In embodiments, at least some number of WBCs may be counted before they have fully settled. For example, in embodiments, images may be captured while WBCs are in the process of settling but have not fully settled to the bottom of the sample chamber. If the depth of field is sufficient, WBCs across a range of depths will appear acceptably in focus in the images. Such images may include brightfield images and/or fluorescence image, and WBCs may be detected in the images by object detection and/or image analytics in the manner described above herein. When a requisite number of WBCs has been counted (e.g., 1,000 WBCs), the imaging operation may be stopped. By capturing images while a constituent of interest is settling, the total amount of time needed to count a requisite number of WBCs may be reduced, or a greater number of WBCs may be counted within a reasonable amount of time.

Accordingly, aspects have been described for capturing fields of view containing WBCs and RBCs in a single analysis and counting a requisite number of WBCs and/or detecting various WBC morphologies. The descriptions above are merely examples. In embodiments, rather than counting 1,000 WBCs, the operations can define a maximum number of fields of view and/or a maximum number of images to be captured. Such maximum numbers of fields of view and/or images may be set to values that would generally yield at least 1,000 WBCs. However, if a biological sample contains fewer WBC than average, then the captured images may yield less than 1,000 WBCs.

The following provides an example of counting and/or evaluating other constituents, as well, in the single analysis, such as RBCs and platelets, among other possible constituents. In the following example, the sample chamber includes a first region having a 100-micrometer depth dimension, a second region having a 200-micrometer depth dimension, and a third region having a 400-micrometer depth dimension. The biological sample will be mixed with a diluent to a particular dilution ratio. The regions are connected and are filled with the same mixture, so the same dilution ratio will be present in all regions. Initially, the mixture will be properly mixed and will have similar density throughout the sample chamber.

In accordance with aspects of the present disclosure, an approach is to evaluate RBCs in the first region having the shallowest depth dimension (e.g., 100-micrometer depth dimension) and then evaluate the mixture in the regions having larger depth dimensions to allow more settling to occur in those regions. A consideration is that the number of RBC in the regions having larger depth dimensions could completely occlude the imaging system since they will be concentrated along with the WBC in the sample. Due to the faster settling rate of WBC compared to RBC, however, WBCs are more likely to be on the bottom of the cartridge and therefore visible to an inverted imaging system, such as that shown in FIG. 4. In addition, if nucleic acid stains are included in the reagents and fluorescence imaging is used, then the RBC can be made to be invisible since they would not fluoresce. Moreover, dilution ratio can be controlled such that the density and number of RBCs would not be overwhelming.

When the cells in the cartridge are allowed to settle, by gravity and waiting or other mechanical means to get them to settle to the bottom, imaging can be performed at the bottom of the cartridge where the cells are effectively concentrated from the entire vertical stack of fluid. Settling will occur at a predictable rate based on the specific gravity of the diluted sample liquid phase and the density of each cell in the sample. Commonly, RBC will settle at a rate of about 1-micrometer per second in reagents that try to preserve cells in an isotonic condition. If natural settling is allowed by gravity, then the majority of cells can then be expected to settle at the constant rate and the wait time before imaging can be calculated based on the depth dimension.

For a 100-micrometer depth dimension, a 200-micrometer depth dimension, and a 400-micrometer depth dimension, settling at 1-micrometer per second results in full settling times of 1 minute and 40 seconds, 3 minutes and 20 seconds, and 6 minutes and 40 seconds, respectively. The imaging device can image the 100-micrometer region first while cells are still settling in the other regions. In the 100-micrometer region, RBC and platelets can be counted and evaluated without risk of crowding. The 200-micrometer region will then be settled and can be imaged and analyzed for cases of anemia where there is an unnaturally low RBC value. The 400-micrometer region will then be settled and RBC will not be analyzed in this region since they will be naturally crowded, but WBC can be evaluated. WBC can be evaluated in all three regions to maximize the number of WBC detected, with a goal of nominally detecting and counting at least 1,000 WBCs.

The 400-micrometer region may impose special conditions because the RBC population will cause crowding. When fluorescence is used, WBC and platelets will fluoresce, while RBC will not (note that immature RBC will fluoresce, but these will not be at the same concentration as mature RBC and will not cause a problem with crowding). This means that in the 400-micrometer region, fluorescence imaging can be used to optically exclude RBC and the remaining cells will no longer crowd the image. As a result of this configuration, WBC will be visible to the imaging device, and RBC do not interfere or cause crowding interference. Indeed, experimentation has found that RBC tend to leave a small space around the settled WBC and are more likely to group up as a population of RBC rather than lean on the settled WBC.

This example approach provides a way to use a single cartridge with multiple depths and with a single dilution value so that RBC, WBC, and platelets can all be evaluated, counted, and analyzed for morphologic changes. Fluorescence imaging in the deeper region is used to optically erase the RBC from the image and support clear WBC evaluation. The approach will decrease the total time needed for cell settling and decrease number of fields of view needed to capture the requisite number of each cell type.

The example is merely illustrative. Other depth dimensions, dilution ratios, and requisite count of WBC are contemplated to be within the scope of the present disclosure.

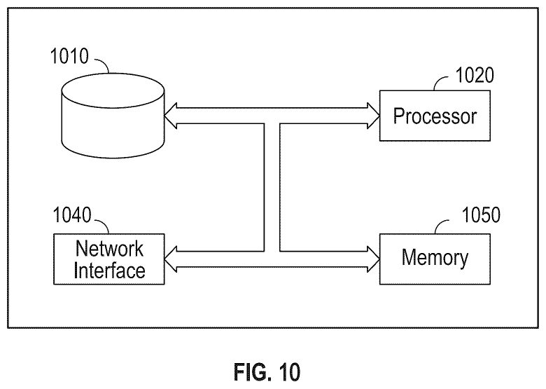

Referring now to FIG. 10, there is shown a block diagram of example components of a point-of-care apparatus at a veterinary facility. The point-of-care apparatus includes an electronic storage 1010, a processor 1020, a network interface 1040, and a memory 1050. The various components may be communicatively coupled with each other. The processor 1020 may be and may include any type of processor, such as a single-core central processing unit (CPU), a multi-core CPU, a microprocessor, a digital signal processor (DSP), a System-on-Chip (SoC), or any other type of processor. The memory 1050 may be a volatile type of memory, e.g., RAM, or a non-volatile type of memory, e.g., NAND flash memory. The memory 1050 includes processor-readable instructions that are executable by the processor 1020 to cause the apparatus to perform various operations, including those mentioned herein, such as the operations shown and described in connection with FIGS. 4 and 7, and/or applying machine learning models or image analytics, among others.

The electronic storage 1010 may be and include any type of electronic storage used for storing data, such as hard disk drive, solid state drive, and/or optical disc, among other types of electronic storage. The electronic storage 1010 stores processor-readable instructions for causing the apparatus to perform its operations and stores data associated with such operations, such as storing data relating to computations and storing captured images, among other data. The network interface 1040 may implement wireless networking technologies and/or wired networking technologies.

The components shown in FIG. 10 are merely examples, and persons skilled in the art will understand that a coordination system includes other components not illustrated and may include multiples of any of the illustrated components. Such and other embodiments are contemplated to be within the scope of the present disclosure.

The above-described embodiments can be expressed in the following numbered aspects:
Aspect A1. An apparatus for analyzing a biological sample, the apparatus comprising:
   at least one processor; and
   at least one memory storing instructions which, when executed by the at least one processor, cause the apparatus at least to perform:
      focusing an imaging device into a sample chamber, the sample chamber having a bottom wall that is at least translucent and through which the imaging device is configured to image an inside of the sample chamber;
      determining that the sample chamber contains at least a diluent and a biological sample comprising red blood cells and white blood cells; and
      controlling the imaging device to capture a plurality of images, the plurality of images comprising at least one of:
         images of a first number of fields of view containing at least a portion of the sample chamber, or
         images of a second number of fields of view containing at least a portion of the sample chamber, wherein the second number is different from the first number,
         wherein the first number of fields of view and the second number of fields of view correspond to at least one of:
         different sample chamber depth dimensions, or
         different ratios of volume of the diluent to volume of the biological sample.
Aspect A2. The apparatus of Aspect A1, wherein the sample chamber comprises a first region having a first depth dimension and a second region having a second depth dimension greater than the first depth dimension,
   wherein the images of the first number of fields of view is captured in the first region having the first depth dimension, and
   wherein the images of the second number of fields of view is captured in the second region having the second depth dimension.
Aspect A3. The apparatus of Aspect A2, wherein the second number of fields of view is less than the first number of fields of view.
Aspect A4. The apparatus of Aspect A2 or Aspect A3, wherein the sample chamber comprises a third region having a third depth dimension greater than the second depth dimension,
   wherein the instructions, when executed by the at least one processor, further cause the apparatus at least to perform:
      controlling the imaging device to capture images of a third number of fields of view containing at least a portion of the third region having the third depth dimension,
      wherein the third number of fields of view is less than the second number of fields of view and is less than the first number of fields of view.
Aspect A5. The apparatus of Aspect A4, wherein in the controlling the imaging device, the instructions, when executed by the at least one processor, further cause the apparatus at least to perform:
   controlling the imaging device to capture the images of the second number of fields of view after capturing the images of the first number of fields of view and to capture the images of the third number of fields of view after capturing the images of the second number of fields of view.
Aspect A6. The apparatus of Aspect A1, wherein the first number of fields of view corresponds to a first ratio of volume of the diluent to volume of the biological sample,
   wherein the second number of fields of view corresponds to a second ratio of volume of the diluent to volume of the biological sample,
   wherein the first ratio is greater than the second ratio, and
   wherein the first number of fields of view is greater than the second number of fields of view.
Aspect A7. The apparatus of any of the preceding Aspects A1-A6, wherein the images of the first number of fields of view comprise images of at least one field of view containing at least a portion of a bottom of the sample chamber, wherein the bottom of the sample chamber is in focus, and
   wherein the images of the second number of fields of view comprise images of at least one field of view containing at least a portion of the bottom of the sample chamber, wherein the bottom of the sample chamber is in focus.
Aspect A8. The apparatus of any of the preceding Aspects A1-A7, wherein the images of the first number of fields of view comprise images focused on a plurality of depths in the sample chamber.
Aspect A9. The apparatus of any of the preceding Aspects A1-A8, wherein the images of the second number of fields of view comprise images focused on a plurality of depths in the sample chamber.
Aspect A10. The apparatus of any of the preceding Aspects A1-A9, wherein the instructions, when executed by the at least one processor, cause the apparatus at least to perform:
   detecting, using an object detection machine learning model, white blood cells in the plurality of images; and
   stopping image capturing by the imaging device when a number of detected white blood cells satisfies a configurable count threshold.
Aspect B1. A method for analyzing a biological sample, the method comprising:
   focusing an imaging device into a sample chamber, the sample chamber having a bottom wall that is at least translucent and through which the imaging device is configured to image an inside of the sample chamber;
   determining that the sample chamber contains at least a diluent and a biological sample comprising red blood cells and white blood cells; and
   controlling the imaging device to capture a plurality of images, the plurality of images comprising at least one of:
      images of a first number of fields of view containing at least a portion of the sample chamber, or
      images of a second number of fields of view containing at least a portion of the sample chamber, wherein the second number is different from the first number,
      wherein the first number of fields of view and the second number of fields of view correspond to at least one of:
         different sample chamber depth dimensions, or
         different ratios of volume of the diluent to volume of the biological sample.
Aspect B2. The method of Aspect B1, wherein the sample chamber comprises a first region having a first depth dimension and a second region having a second depth dimension greater than the first depth dimension,
   wherein the images of the first number of fields of view is captured in the first region having the first depth dimension, and
   wherein the images of the second number of fields of view is captured in the second region having the second depth dimension.
Aspect B3. The method of Aspect B2, wherein the second number of fields of view is less than the first number of fields of view.
Aspect B4. The method of Aspect B2 or Aspect B3, wherein the sample chamber comprises a third region having a third depth dimension greater than the second depth dimension,
   the method further comprising:
      controlling the imaging device to capture images of a third number of fields of view containing at least a portion of the third region having the third depth dimension,
      wherein the third number of fields of view is less than the second number of fields of view and is less than the first number of fields of view.
Aspect B5. The method of Aspect B4, wherein the controlling the imaging device comprises:
   controlling the imaging device to capture the images of the second number of fields of view after capturing the images of the first number of fields of view and to capture the images of the third number of fields of view after capturing the images of the second number of fields of view.
Aspect B6. The method of Aspect B1, wherein the first number of fields of view corresponds to a first ratio of volume of the diluent to volume of the biological sample,
   wherein the second number of fields of view corresponds to a second ratio of volume of the diluent to volume of the biological sample,
   wherein the first ratio is greater than the second ratio, and
   wherein the first number of fields of view is greater than the second number of fields of view.
Aspect B7. The method of any of the preceding Aspects B1-B6, wherein the images of the first number of fields of view comprise images of at least one field of view containing at least a portion of a bottom of the sample chamber, wherein the bottom of the sample chamber is in focus, and
   wherein the images of the second number of fields of view comprise images of at least one field of view containing at least a portion of the bottom of the sample chamber, wherein the bottom of the sample chamber is in focus.
Aspect B8. The method of any of the preceding Aspects B1-B7, wherein the images of the first number of fields of view comprise images focused on a plurality of depths in the sample chamber.
Aspect B9. The method of any of the preceding Aspects B1-B8, wherein the images of the second number of fields of view comprise images focused on a plurality of depths in the sample chamber.
Aspect B10. The method of any of the preceding Aspects B1-B9, further comprising:
   detecting, using an object detection machine learning model, white blood cells in the plurality of images; and
   stopping image capturing by the imaging device when a number of detected white blood cells satisfies a configurable count threshold.
Aspect C1. An processor-readable medium storing instructions which, when executed by one or more processors of an apparatus, causes the apparatus at least to perform:
   focusing an imaging device into a sample chamber, the sample chamber having a bottom wall that is at least translucent and through which the imaging device is configured to image an inside of the sample chamber;
   determining that the sample chamber contains at least a diluent and a biological sample comprising red blood cells and white blood cells; and
   controlling the imaging device to capture a plurality of images, the plurality of images comprising at least one of:
      images of a first number of fields of view containing at least a portion of the sample chamber, or
      images of a second number of fields of view containing at least a portion of the sample chamber, wherein the second number is different from the first number,
      wherein the first number of fields of view and the second number of fields of view correspond to at least one of:
         different sample chamber depth dimensions, or
         different ratios of volume of the diluent to volume of the biological
Aspect C2. The processor-readable medium of Aspect C1, wherein the sample chamber comprises a first region having a first depth dimension and a second region having a second depth dimension greater than the first depth dimension,
   wherein the images of the first number of fields of view are captured in the first region having the first depth dimension, and
   wherein the images of the second number of fields of view are captured in the second region having the second depth dimension.
Aspect C3. The processor-readable medium of Aspect C2, wherein the second number of fields of view is less than the first number of fields of view.
Aspect C4. The processor-readable medium of Aspect C2 or Aspect C3, wherein the sample chamber comprises a third region having a third depth dimension greater than the second depth dimension,
   wherein the instructions, when executed by the at least one processor, further cause the apparatus at least to perform:
      controlling the imaging device to capture images of a third number of fields of view containing at least a portion of the third region having the third depth dimension,
      wherein the third number of fields of view is less than the second number of fields of view and is less than the first number of fields of view.
Aspect C5. The processor-readable medium of Aspect C4, wherein in the controlling the imaging device, the instructions, when executed by the at least one processor, further cause the apparatus at least to perform:
   controlling the imaging device to capture the images of the second number of fields of view after capturing the images of the first number of fields of view and to capture the images of the third number of fields of view after capturing the images of the second number of fields of view.
Aspect C6. The processor-readable medium of Aspect C1, wherein the first number of fields of view corresponds to a first ratio of volume of the diluent to volume of the biological sample,
   wherein the second number of fields of view corresponds to a second ratio of volume of the diluent to volume of the biological sample,
   wherein the first ratio is greater than the second ratio, and
   wherein the first number of fields of view is greater than the second number of fields of view.
Aspect C7. The processor-readable medium of any of the preceding Aspects C1-C6, wherein the images of the first number of fields of view comprise images of at least one field of view containing at least a portion of a bottom of the sample chamber, wherein the bottom of the sample chamber is in focus, and
   wherein the images of the second number of fields of view comprise images of at least one field of view containing at least a portion of the bottom of the sample chamber, wherein the bottom of the sample chamber is in focus.
Aspect C8. The processor-readable medium of any of the preceding Aspects C1-C7, wherein the images of the first number of fields of view comprise images focused on a plurality of depths in the sample chamber.
Aspect C9. The processor-readable medium of any of the preceding Aspects C1-C8, wherein the images of the second number of fields of view comprise images focused on a plurality of depths in the sample chamber.
Aspect C10. The processor-readable medium of any of the preceding Aspects C1-C9, wherein the instructions, when executed by the at least one processor, cause the apparatus at least to perform:
   detecting, using an object detection machine learning model, white blood cells in the plurality of images; and
   stopping image capturing by the imaging device when a number of detected white blood cells satisfies a configurable count threshold.

The embodiments disclosed herein are examples of the disclosure and may be embodied in various forms. For instance, although certain embodiments herein are described as separate embodiments, each of the embodiments herein may be combined with one or more of the other embodiments herein. Specific structural and functional details disclosed herein are not to be interpreted as limiting, but as a basis for the claims and as a representative basis for teaching one skilled in the art to variously employ the present disclosure in virtually any appropriately detailed structure. Like reference numerals may refer to similar or identical elements throughout the description of the figures.

The phrases “in an embodiment,” “in embodiments,” “in various embodiments,” “in some embodiments,” or “in other embodiments” may each refer to one or more of the same or different embodiments in accordance with the present disclosure. A phrase in the form “A or B” means “(A), (B), or (A and B).” A phrase in the form “at least one of A, B, or C” means “(A); (B); (C); (A and B); (A and C); (B and C); or (A, B, and C).”

The systems, devices, and/or servers described herein may utilize one or more processors to receive various information and transform the received information to generate an output. The processors may include any type of computing device, computational circuit, or any type of controller or processing circuit capable of executing a series of instructions that are stored in a memory. The processor may include multiple processors and/or multicore central processing units (CPUs) and may include any type of device, such as a microprocessor, graphics processing unit (GPU), digital signal processor, microcontroller, programmable logic device (PLD), field programmable gate array (FPGA), or the like. The processor may also include a memory to store data and/or instructions that, when executed by the one or more processors, causes the one or more processors (and/or the systems, devices, and/or servers they operate in) to perform one or more methods, operations, and/or algorithms.

Any of the herein described methods, operations, programs, algorithms or codes may be converted to, or expressed in, a programming language or computer program. The terms “programming language” and “computer program,” as used herein, each include any language used to specify instructions to a computer, and include (but is not limited to) the following languages and their derivatives: Assembler, Basic, Batch files, BCPL, C, C+, C++, Delphi, Fortran, Java, JavaScript, machine code, operating system command languages, Pascal, Perl, PL1, Python, scripting languages, Visual Basic, metalanguages which themselves specify programs, and all first, second, third, fourth, fifth, or further generation computer languages. Also included are database and other data schemas, and any other meta-languages. No distinction is made between languages which are interpreted, compiled, or use both compiled and interpreted approaches. No distinction is made between compiled and source versions of a program. Thus, reference to a program, where the programming language could exist in more than one state (such as source, compiled, object, or linked) is a reference to any and all such states. Reference to a program may encompass the actual instructions and/or the intent of those instructions.

It should be understood that the foregoing description is only illustrative of the present disclosure. Various alternatives and modifications can be devised by those skilled in the art without departing from the disclosure. Accordingly, the present disclosure is intended to embrace all such alternatives, modifications and variances. The embodiments described with reference to the attached drawing figures are presented only to demonstrate certain examples of the disclosure. Other elements, steps, methods, and techniques that are insubstantially different from those described above and/or in the appended claims are also intended to be within the scope of the disclosure.

What is claimed:

1. An apparatus for analyzing a biological sample, the apparatus comprising:
at least one processor; and
at least one memory storing instructions which, when executed by the at least one processor, cause the apparatus at least to perform:
focusing an imaging device into a sample chamber, the sample chamber having a bottom wall that is at least translucent and through which the imaging device is configured to image an inside of the sample chamber;
determining that the sample chamber contains at least a diluent and a biological sample comprising red blood cells and white blood cells; and
controlling the imaging device to capture a plurality of images, the plurality of images comprising at least one of:
images of a first number of fields of view containing at least a portion of the sample chamber, or
images of a second number of fields of view containing at least a portion of the sample chamber, wherein the second number is different from the first number,
wherein the first number of fields of view and the second number of fields of view correspond to at least one of:
different sample chamber depth dimensions, or
different ratios of volume of the diluent to volume of the biological sample.

2. The apparatus of claim 1, wherein the sample chamber comprises a first region having a first depth dimension and a second region having a second depth dimension greater than the first depth dimension,
wherein the images of the first number of fields of view are captured in the first region having the first depth dimension, and
wherein the images of the second number of fields of view are captured in the second region having the second depth dimension.

3. The apparatus of claim 2, wherein the second number of fields of view is less than the first number of fields of view.

4. The apparatus of claim 2, wherein the sample chamber comprises a third region having a third depth dimension greater than the second depth dimension,
wherein the instructions, when executed by the at least one processor, further cause the apparatus at least to perform:
controlling the imaging device to capture images of a third number of fields of view containing at least a portion of the third region having the third depth dimension,
wherein the third number of fields of view is less than the second number of fields of view and is less than the first number of fields of view.

5. The apparatus of claim 4, wherein in the controlling the imaging device, the instructions, when executed by the at least one processor, further cause the apparatus at least to perform:
controlling the imaging device to capture the images of the second number of fields of view after capturing the images of the first number of fields of view and to capture the images of the third number of fields of view after capturing the images of the second number of fields of view.

6. The apparatus of claim 1, wherein the first number of fields of view corresponds to a first ratio of volume of the diluent to volume of the biological sample,
wherein the second number of fields of view corresponds to a second ratio of volume of the diluent to volume of the biological sample,
wherein the first ratio is greater than the second ratio, and
wherein the first number of fields of view is greater than the second number of fields of view.

7. The apparatus of claim 1, wherein the images of the first number of fields of view comprise images of at least one field of view containing at least a portion of a bottom of the sample chamber, wherein the bottom of the sample chamber is in focus,
wherein the images of the second number of fields of view comprise images of at least one field of view containing at least a portion of the bottom of the sample chamber, wherein the bottom of the sample chamber is in focus,
wherein the images of the first number of fields of view comprise images focused on a plurality of depths in the sample chamber, and
wherein the images of the second number of fields of view comprise images focused on a plurality of depths in the sample chamber.

8. A method for analyzing a biological sample, the method comprising:
focusing an imaging device into a sample chamber, the sample chamber having a bottom wall that is at least translucent and through which the imaging device is configured to image an inside of the sample chamber;
determining that the sample chamber contains at least a diluent and a biological sample comprising red blood cells and white blood cells; and
controlling the imaging device to capture a plurality of images, the plurality of images comprising at least one of:
images of a first number of fields of view containing at least a portion of the sample chamber, or
images of a second number of fields of view containing at least a portion of the sample chamber, wherein the second number is different from the first number,
wherein the first number of fields of view and the second number of fields of view correspond to at least one of:
different sample chamber depth dimensions, or
different ratios of volume of the diluent to volume of the biological sample.

9. The method of claim 8, wherein the sample chamber comprises a first region having a first depth dimension and a second region having a second depth dimension greater than the first depth dimension,
wherein the images of the first number of fields of view are captured in the first region having the first depth dimension, and
wherein the images of the second number of fields of view are captured in the second region having the second depth dimension.

10. The method of claim 9, wherein the second number of fields of view is less than the first number of fields of view.

11. The method of claim 9, wherein the sample chamber comprises a third region having a third depth dimension greater than the second depth dimension,
the method further comprising:
controlling the imaging device to capture images of a third number of fields of view containing at least a portion of the third region having the third depth dimension,
wherein the third number of fields of view is less than the second number of fields of view and is less than the first number of fields of view.

12. The method of claim 11, wherein the controlling the imaging device comprises:
controlling the imaging device to capture the images of the second number of fields of view after capturing the images of the first number of fields of view and to capture the images of the third number of fields of view after capturing the images of the second number of fields of view.

13. The method of claim 8, wherein the first number of fields of view corresponds to a first ratio of volume of the diluent to volume of the biological sample,
wherein the second number of fields of view corresponds to a second ratio of volume of the diluent to volume of the biological sample,
wherein the first ratio is greater than the second ratio, and
wherein the first number of fields of view is greater than the second number of fields of view.

14. The method of claim 8, wherein the images of the first number of fields of view comprise images of at least one field of view containing at least a portion of a bottom of the sample chamber, wherein the bottom of the sample chamber is in focus,
wherein the images of the second number of fields of view comprise images of at least one field of view containing at least a portion of the bottom of the sample chamber, wherein the bottom of the sample chamber is in focus,
wherein the images of the first number of fields of view comprise images focused on a plurality of depths in the sample chamber, and
wherein the images of the second number of fields of view comprise images focused on a plurality of depths in the sample chamber.

15. A processor-readable medium storing instructions which, when executed by at least one processor of an apparatus, causes the apparatus at least to perform:
focusing an imaging device into a sample chamber, the sample chamber having a bottom wall that is at least translucent and through which the imaging device is configured to image an inside of the sample chamber;
determining that the sample chamber contains at least a diluent and a biological sample comprising red blood cells and white blood cells; and
controlling the imaging device to capture a plurality of images, the plurality of images comprising at least one of:
images of a first number of fields of view containing at least a portion of the sample chamber, or
images of a second number of fields of view containing at least a portion of the sample chamber, wherein the second number is different from the first number,
wherein the first number of fields of view and the second number of fields of view correspond to at least one of:
different sample chamber depth dimensions, or
different ratios of volume of the diluent to volume of the biological sample.

16. The processor-readable medium of claim 15, wherein the sample chamber comprises a first region having a first depth dimension and a second region having a second depth dimension greater than the first depth dimension,
wherein the images of the first number of fields of view are captured in the first region having the first depth dimension, and
wherein the images of the second number of fields of view are captured in the second region having the second depth dimension.

17. The processor-readable medium of claim 16, wherein the second number of fields of view is less than the first number of fields of view.

18. The processor-readable medium of claim 16, wherein the sample chamber comprises a third region having a third depth dimension greater than the second depth dimension,
wherein the instructions, when executed by the at least one processor, further cause the apparatus at least to perform:
controlling the imaging device to capture a images of a third number of fields of view containing at least a portion of the third region having the third depth dimension,
wherein the third number of fields of view is less than the second number of fields of view and is less than the first number of fields of view.

19. The processor-readable medium of claim 18, wherein in the controlling the imaging device, the instructions, when executed by the at least one processor, further cause the apparatus at least to perform:
controlling the imaging device to capture the images of the second number of fields of view after capturing the images of the first number of fields of view and to capture the images of the third number of fields of view after capturing the images of the second number of fields of view.

20. The processor-readable medium of claim 15, wherein the first number of fields of view corresponds to a first ratio of volume of the diluent to volume of the biological sample,
wherein the second number of fields of view corresponds to a second ratio of volume of the diluent to volume of the biological sample,
wherein the first ratio is greater than the second ratio, and
wherein the first number of fields of view is greater than the second number of fields of view.
